(19) United States
(12) Patent Application Publication
(10) Pub. No.: US 2006/0133961 A1
(43) Pub. Date: Jun. 22, 2006

(54) Bi-material cantilevers with flipped over material sections and structures formed therefrom

(75) Inventors: Si-Hyung Lim, Albany, CA (US); Jongeun Choi, Berkeley, CA (US); Roberto Horowitz, El Cerrito, CA (US); Arun Majumdar, Orinda, CA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: The Regents of the University of California, Oakland, CA (US)

(21) Appl. No.: 11/255,669

(22) Filed: Oct. 20, 2005

Related U.S. Application Data
(60) Provisional application No. 60/621,170, filed on Oct. 21, 2004.

(51) Int. Cl. G01N 33/00

(52) U.S. Cl. 422/88; 422/68.1

(57) ABSTRACT

A structure formed from one or more bi-material cantilevers, having a first portion and a second portion positioned end-to-end, wherein the first portion comprises a first material positioned on top of a second material, and wherein the second portion comprises the second material positioned on top of the first material.

### RELATED APPLICATION

This patent application claims the benefit of the filing date of U.S. Provisional Patent Application Ser. No. 60/621,170, filed Oct. 21, 2004 entitled “Design and Fabrication of a Novel Bimorph Micro-Opto-Mechanical Sensor”, the entire contents of which is hereby expressly incorporated by reference.

### TECHNICAL FIELD

The present invention relates to microcantilevers in general and to microcantilever sensor systems in particular.

### BACKGROUND OF THE INVENTION

Microcanilever temperature sensors have been formed from two layer bi-material cantilevers in the past. In such systems, a first material layer is placed on top of a second material layer. The first and second materials have different thermal expansion coefficients. As a result, when the temperature of the cantilever is increased, the first and second layers will expand at different rates. As a result, the top portion of the cantilever will expand at a rate different from the rate at which the bottom of the cantilever expands. This difference in expansion rates between the two layers causes the cantilever to deflect. By measuring the amount of deflection at the free end of the cantilever, it is possible to accurately determine the temperature of the cantilever.

Unfortunately, in such existing bi-material thermal sensing cantilever systems, the free end of the cantilever becomes angled with respect to the fixed end of the cantilever as the cantilever deflects. As will be explained herein, this limits the degree to which it is possible to build multi-cantilever sensor systems. In addition, light reflected from the free end of the cantilever will be reflected at different angles as the cantilever deflects. Thus, detection of cantilever deflection requires monitoring of the location of a changing path of reflected laser light. As will be shown herein, the present invention overcomes both of these disadvantages.

### SUMMARY OF THE INVENTION

The present invention provides a bi-material cantilever, having: a first portion and a second portion positioned end-to-end, wherein the first portion comprises a first material positioned on top of a second material, and wherein the second portion comprises the second material positioned on top of the first material.

The first and second materials have different thermal expansion coefficients. As a result, the opposite ends of the cantilever remain parallel to one another during deflection of the cantilever. Therefore, the present invention can be assembled into a variety of sensor systems, offering unique advantages as will be shown.

In one optional embodiment, the first material is nitride and the second material is aluminum. However, other materials may equally be used. In preferred embodiments, the first material is optionally deposited on the second material by plasma enhanced chemical vapor deposition or by electron beam evaporation. Again, however, the present invention is again not so limited. Other methods of fabrication may also be used.

In optional structures encompassed by the present invention, a reflective panel may be connected to one end of the cantilever. In optional embodiments, an infra-red radiation absorbing pad may be connected to the reflective panel, or to one of the cantilevers.

In various embodiments of the invention, an electrical path may be disposed along a surface of the bi-material cantilever. In such embodiments, the present invention may be used in a voltage sensor with the current flow through the electrical path varying the temperature of the cantilever.

In various other optional embodiments, a surface of the first material of the bi-material cantilever may be functionalized with one or more probe substances such that when a target substance binds thereto, a change in surface stress occurs on the surface, thereby causing the cantilever to deflect. In such embodiments, the present invention may be used as a chemical or bio-chemical sensor.

The present invention also encompasses a structure formed from a pair of bi-material cantilevers, wherein each bi-material cantilever includes: a first portion and a second portion positioned end-to-end, wherein the first portion of each bi-material cantilever comprises a first material positioned on top of a second material, and wherein the second portion of each bi-material cantilever comprises the second material positioned on top of the first material, and wherein the pair of bi-material cantilevers are connected together.

In various embodiments of the structure, the cantilevers may be connected together end-to-end, or through an intermediate member. Such intermediate member may comprise a small block of material, or it may optionally comprise a device such as a reflective panel. In various embodiments of the invention, the movement of the reflective panel (caused by deflection of the cantilever(s)) can be used to optically measure temperature or voltage changes, or to sense the presence or concentration of a chemical or bio-chemical substance.

When the first and second cantilevers are connected together, they may optionally be connected such that the first portion of one cantilever is connected to the second portion of the other cantilever. In other words, successive cantilevers may be “flipped over” with respect to one another. It is to be understood, however, the present invention is not so limited.

The present invention also provides a variety of sensor systems build from series of bi-material cantilevers assembled together into different structures. In various embodiment, the bi-material cantilevers are connected together at right angles to one another. Alternately, however, they may be positioned parallel to one another.

### BRIEF DESCRIPTION OF THE DRAWINGS

FIG. 1 is a side elevation view of a first embodiment of the present bi-material cantilever prior to cantilever deflection.

FIG. 2 is a view corresponding to FIG. 1 after cantilever deflection.

FIG. 3 is a side elevation view of a second embodiment of the present bi-material cantilever prior to cantilever deflection.

FIG. 4 is a view corresponding to FIG. 3 after cantilever deflection.

FIG. 5 is a side elevation view of a third embodiment of the present bi-material cantilever prior to cantilever deflection.

FIG. 6 is a view corresponding to FIG. 5 after cantilever deflection.

FIG. 7 is a side elevation view of a pre-existing bi-material cantilever prior to cantilever deflection with a laser beam directed at the free end of the cantilever.

FIG. 8 is a view corresponding to FIG. 7 after cantilever deflection showing the laser beam reflected off of the free end of the cantilever.

FIG. 9 is a side elevation view of the present bi-material cantilever prior to cantilever deflection with a laser beam directed at the free end of the cantilever.

FIG. 10 is a view corresponding to FIG. 9 after cantilever deflection showing the laser beam reflected off of the free end of the cantilever.

FIG. 11 is a perspective view of a structure in which two parallel bi-material cantilevers are connected together.

FIG. 12 is a perspective view of a structure in which three parallel bi-material cantilevers are connected together.

FIG. 13 is a perspective view of a structure comprising a series of bi-material cantilevers, an infra-red radiation absorbing pad and a reflective panel prior to cantilever deflection.

FIG. 14 is a perspective view of the structure of FIG. 13 after cantilever deflection.

FIG. 15 is a perspective view of a structure comprising a series of bi-material cantilevers and a reflective panel prior to cantilever deflection.

FIG. 16 is a perspective view of the structure of FIG. 15 after cantilever deflection.

FIG. 17 is a top plan view of a structure for sensing infra-red radiation comprising a series of bi-material cantilevers and a reflective panel prior to cantilever deflection.

FIG. 18 is a side elevation view of the structure of FIG. 17, prior to cantilever deflection.

FIG. 19 is a view corresponding to FIG. 18, after cantilever deflection.

FIG. 20 is a top plan view of a structure comprising a series of bi-material cantilevers and a reflective panel, wherein the reflective panel has three degrees of freedom of movement.

FIG. 21 is a side elevation view similar to FIG. 1, but with a surface of a first material functionalized with a probe substance, prior to cantilever deflection.

FIG. 22 is a view corresponding to FIG. 21 with a target substance binding to the probe substance, causing a change in surface stresses in the cantilever, resulting in cantilever deflection.

### DETAILED DESCRIPTION OF THE DRAWINGS

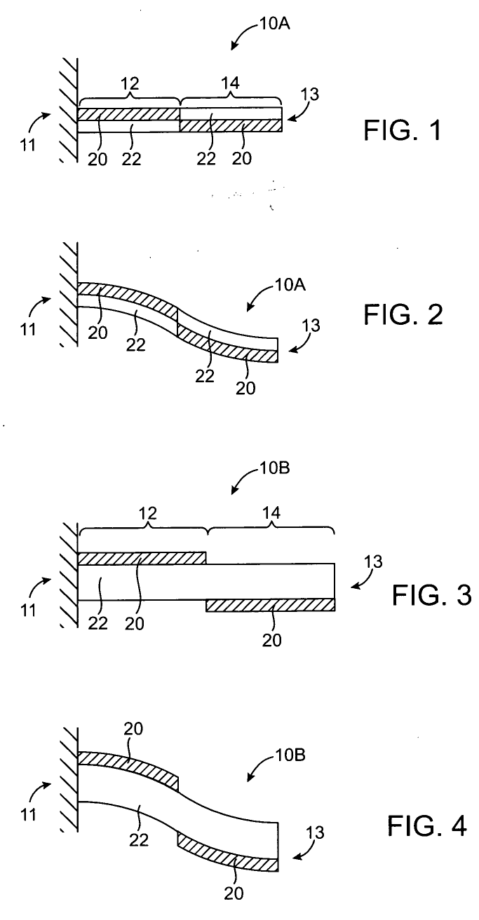

FIGS. 1, 3 and 5 show side elevation views of three embodiments of the present bi-material cantilever prior to deflection. FIGS. 2, 4, and 6 show corresponding side elevation views after cantilever deflection, revealing unique properties of the present invention.

First, as shown in FIG. 1, a bi-material cantilever 10A is provided. Cantilever 10A has a first portion 12 and a second portion 14 positioned end-to-end. First portion 12 comprises a first material 20 positioned on top of a second material 22. Second portion 14 comprises the second material 22 positioned on top of the first material 20.

In accordance with the present invention, first material 20 and second material 22 have different thermal expansion coefficients. As illustrated, first material 20 has a greater thermal expansion co-efficient than second material 22. As a result, when cantilever 10A is heated, first material 20 will expand more than second material 22, causing the cantilever to deflect to the position as shown in FIG. 2.

As can be seen by comparing FIGS. 1 and 2, a unique property of the present invention is that its opposite ends 11 and 13 remain parallel to one another regardless of the degree of cantilever deflection. IE: free end 13 reamains parallel to fixed end 11. This particular advantage of the present invention will be explained fully in various embodiments of the invention described herein.

As show in FIG. 1, first material 20 in first portion 12 of cantilever 10A abuts second material 22 in second portion 14 of cantilever 10A. In preferred embodiments of the present invention, the first and second portions 12 and 14 of cantilever 10A are of equal length. In addition, as shown in FIGS. 1 and 2, first and second portions 12 and 14 may each comprises one half of the length of cantilever 10A.

In optional preferred embodiments, first material 20 is nitride, and second material 22 is aluminum. It is to be understood that the present invention is not so limited and that other suitable materials be readily substituted.

As shown in FIG. 3, second material 22 may alternately comprise a continuous strip of material through the entire length of cantilever 10B. Thus, second material 22 in first portion 12 of cantilever 10B is continuous with second material 22 in second portion 14 of cantilever 10B. First material 20 is then deposited or otherwise fabricated onto top and bottom portions of the continuous sheet of second material 22, as shown.

In one embodiment, first material 20 may be deposited on second material 22 by plasma enhanced chemical vapor deposition or by electron beam evaporation. However, the present invention is not so limited. Other fabrication techniques may be used.

As shown in FIG. 4, the deflection of cantilever 10B also results in opposite ends 11 and 13 remaining parallel to one another regardless of the degree of cantilever deflection. (Similar to FIG. 2).

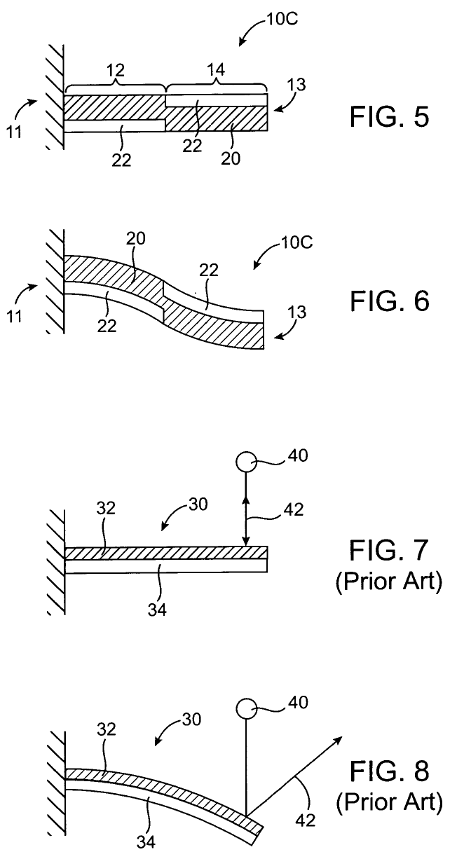

As shown in FIG. 5, cantilever 10C may alternately comprise a continuous first material 20 with portions of the cantilever formed from second material 22, as shown. In this embodiment, the two sections of second material 22 are spaced apart by a portion of first material 20, as shown. In this embodiment, the length/amount of second material 22 in first portion 12 is the same as in second portion 14.

As shown in FIG. 6, deflection of cantilever 10C also results in opposite ends 11 and 13 remaining parallel to one another regardless of the degree of cantilever deflection. (Similar to FIGS. 2 and 4).

FIGS. 7 and 8 show deflection of a standard pre-existing bi-material cantilever. As seen in FIG. 7, cantilever 30 has a top layer 32 made from a first material and a bottom layer 34 made from a second material. Top layer 32 has a greater thermal coefficient of expansion than bottom layer 34. As a result, when cantilever 30 is heated, it deflects from the position shown in FIG. 7 to the position shown in FIG. 8. A laser 40 directs a laser beam 42 at the free end of cantilever.

As shown in FIG. 7, laser beam 42 is reflected back toward laser 40 when cantilever 30 is not deflected. As shown in FIG. 8, laser beam 42 is reflected away from laser 40 when cantilever 30 is deflected.

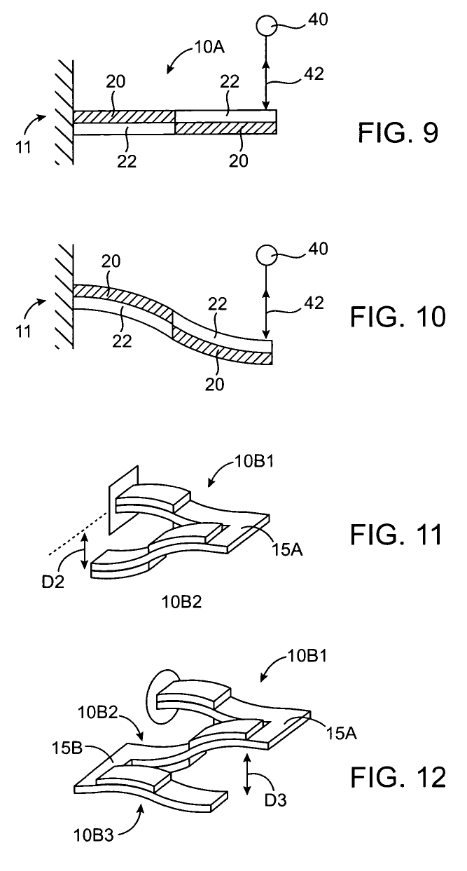

In contrast, FIGS. 9 and 10 show deflection of the present bi-material cantilever. As seen in FIG. 9, laser 40 directs a laser beam 42 at the free end of cantilever 10A, with laser beam 42 being reflected back toward laser 40. As shown in FIG. 9, laser beam 42 is still reflected back toward laser 40 when cantilever 10A deflects.

FIG. 11 shows an exemplary sensor structure formed from two parallel bi-material cantilevers 10B1 and 10B2. Cantilevers 10B1 and 10B2 are connected together through an intermediate member 15A. One first end of cantilever 10B1 is fixed and one end of cantilever 10B2 is free. As can be seen, when cantilevers 10B1 and 10B2 each deflect by an identical distance D, the free end of cantilever 10B2 moves a distance 2D.

FIG. 12 shows a similar exemplary sensor structure formed from three parallel bi-material cantilevers 10B1, 10B2 and 10B3. Cantilevers 10B1 and 10B2 are connected together through an intermediate member 15A. Cantilevers 10B2 and 10B3 are connected together through an intermediate member 151B. One first end of cantilever 10B1 is fixed and one end of cantilever 10B3 is free. As can be seen, when cantilevers 10B1, 10B2 and 10B3 each deflect by an identical distance D, the free end of cantilever 10B2 moves a distance 3D.

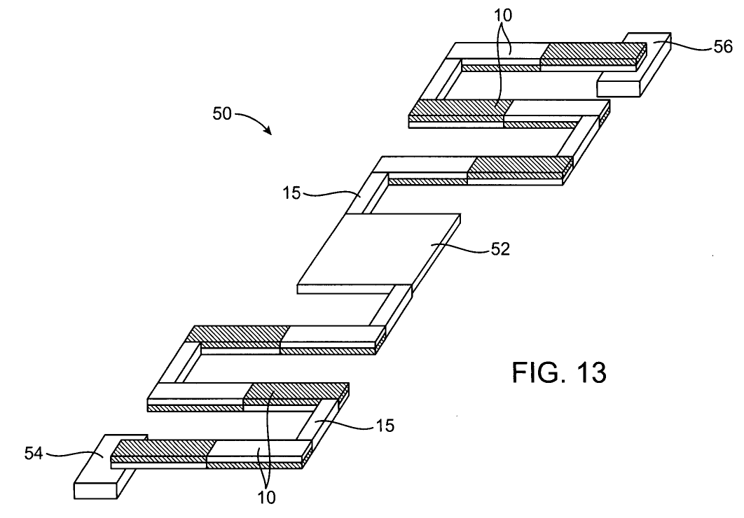

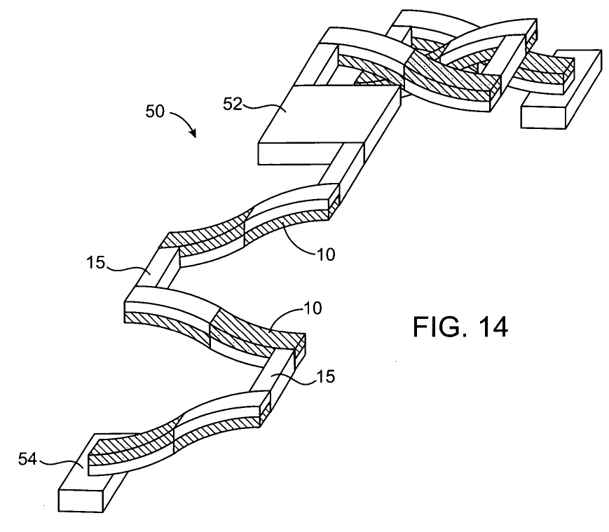

FIGS. 13 and 14 show actuation of a first embodiment of a voltage sensor 50, as follows. In FIG. 13, a plurality of cantilevers 10 are positioned parallel to one another, with successive cantilevers 10 connected through intermediate portions 15. A reflective panel 52 is positioned at the center of the structure, as shown. Electrical contacts 54 and 56 are positioned at opposite ends of the sensor 50.

Consequently, as shown in FIG. 14, when a voltage is applied to contacts 54 and 56, a current passes through cantilevers 10, warming the cantilevers and causing them to deflect. As a result of the cumulative deflections of all the cantilevers 10, the reflective panel 52 moves to the position shown. As can be seen, the various positions to which reflective panel 52 moves when cantilevers 10 deflect are all parallel to one another. In operation, the amount of voltage passing through sensor 50 is proportional to the vertical movement of reflective panel 52. Thus, by sensing the movement of reflective panel 52, the voltage passing through sensor 50 can be determined.

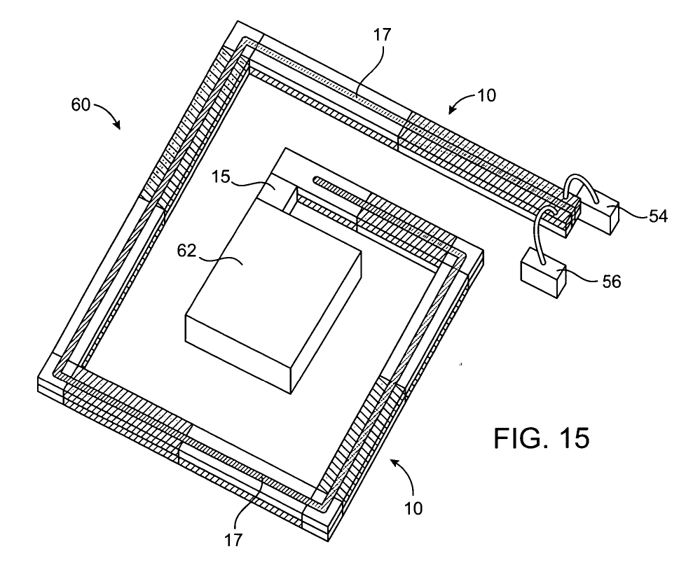

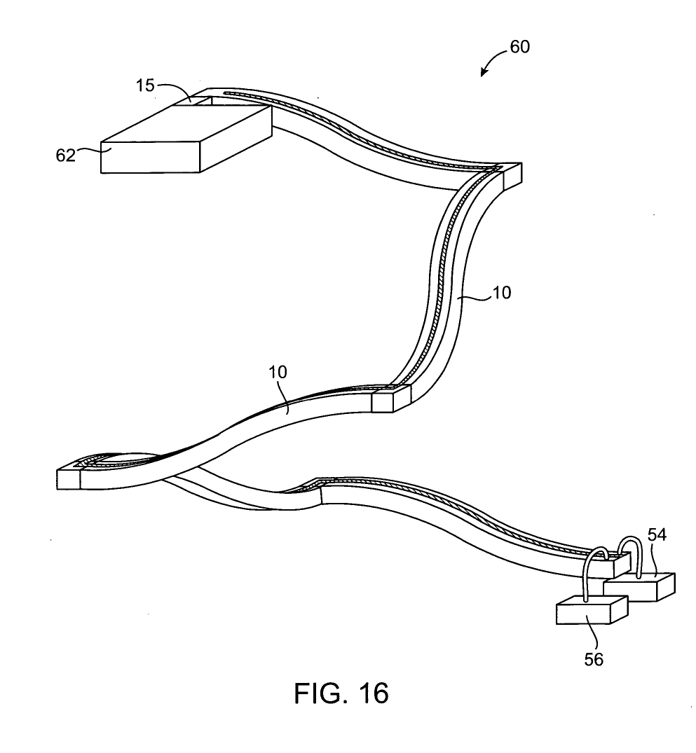

FIGS. 15 and 16 show another embodiment of a voltage sensor, as follows. Voltage sensor 60 comprises reflective panel 52 connected to a series of cantilevers 10. Cantilevers 10 are in turn connected together by intermediate members 15. Electrical contacts 54 and 56 are also provided. In sensor 60, each of cantilevers 10 has a strip of electrically isolating material 17 running therealong. As a result, electrical contacts 54 and 56 can be connected to cantilevers 10 as shown with current passing along paths through opposite sides of the individual cantilevers. An advantage of this design is that the amount of vertical movement of reflective panel 52 will equal the cumulative deflection of all of the individual cantilevers 10. In an alternate embodiment, the current does not pass along through opposite sides of the individual cantilevers. Instead, current is conducted in paths along the top and bottom surfaces of the cantilevers.

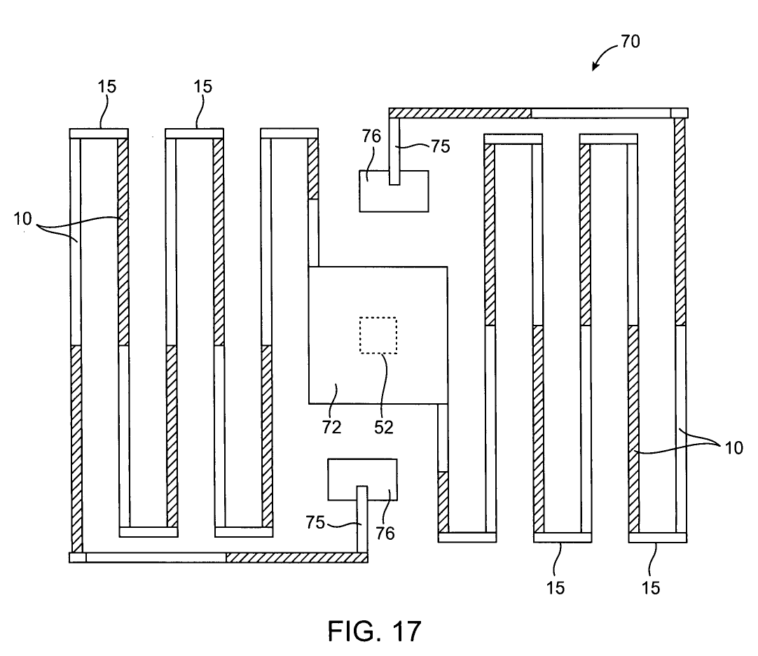

FIGS. 17 to 19 show an embodiment of an infra-red sensor, as follows. Infra-red sensor 70 comprises a series of cantilevers 10 connected together by intermediate portions 15. An infra-red radiation absorbing pad 72 is positioned above reflective panel 52. Laser 40 is positioned below reflective panel 52, directing laser beam 42 toward reflective panel 52, as shown.

As also seen in FIG. 17, a pair of thermal isolation elements 75 are connected to opposite ends of the series of cantilevers 10. At the ends of thermal isolation elements 75 are contact pads 76. Contact pads 76 are used for mounting sensor 70 to a support or frame (not shown). Thermal isolation elements 75 minimize heat transfer between cantilevers 10 and the support or frame (not shown).

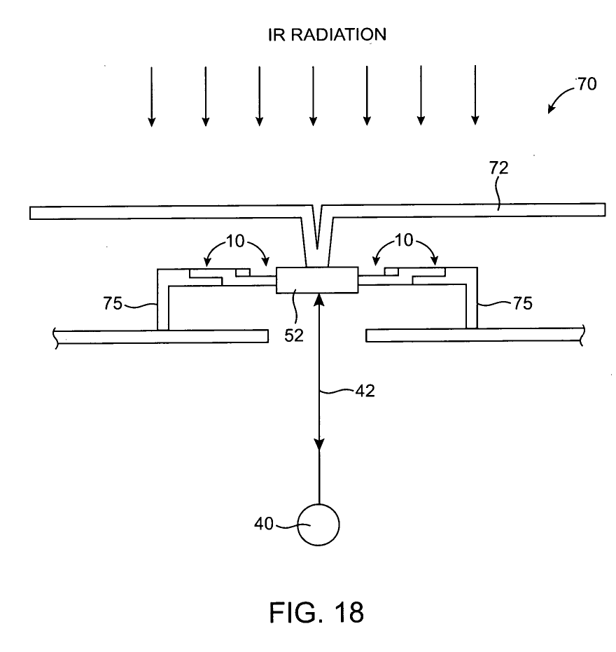

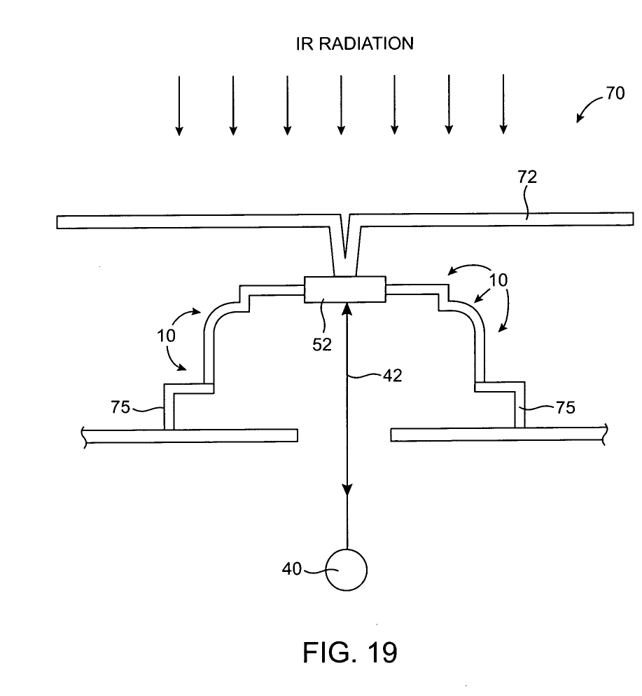

Operation of sensor 70 is shown in FIGS. 18 and 19 as follows. In FIG. 18, infra-red radiation is directed down onto infra-red radiation absorbing pad 72. This heat is then conducted through reflective panel 52 and into cantilevers 10. As a result, cantilevers 10 deflect to the position shown in FIG. 18. As can be seen, such deflection moves reflective panel 52 upwardly. The distance reflective panel 52 moves is proportional to the degree of cantilever deflection (which is in turn proportional to the temperature of the cantilevers). As a result, by sensing the movement of reflective panel 52, the amount of infra-red radiation contacting absorbing pad 72 can be determined.

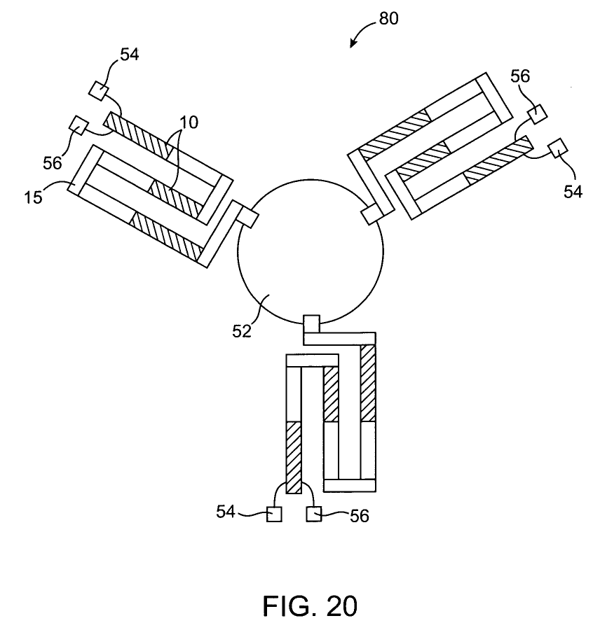

FIG. 20 shows an embodiment of a voltage sensor 80 in which a reflective panel 52 is supported by three cantilever structures 81, 82 and 83. Each cantilever structure 81, 82 and 83 is formed from a plurality of cantilevers arranged in a geometry similar to that shown in FIGS. 13 and 14, but using a strip of electrical isolation material 17 on cantilevers 10 similar to the arrangement shown in FIGS. 15 and 16. Each cantilever structure 81, 82 and 83 has a pair of electrical contacts 54 and 56 connected thereto. As a result, when a voltage is applied across any particular pair of electrical contacts 54 and 56, the cantilevers in cantilever structure 81, 82 or 83 will deflect. A particular advantage of sensor 80 is that different voltages may be applied to each of cantilever structures 81, 82 and 83, causing them to deflect to different degrees. As a result, reflective panel 52 is moveable in three degrees of freedom. As a result, a laser beam (not shown) reflected off of reflective panel 52 can be reflected in various directions depending upon the particular orientation of reflective panel 52 in space. Relative deflection of each of cantilever structures 81, 82 and 83 with respect to one another with cause tilting of reflective panel 52, thus moving the path of a reflected laser beam in two dimensions. Moreover, when all three cantilever structures 81, 82 and 83 deflect by the same amount, such deflection can be measured by determining the movement of reflective panel 52. Therefore, by determining the particular path of a laser beam reflected off of reflective panel 52, it is possible to determine the various voltages across each of the three pairs of electrical contacts 54 and 56 for each of the respective cantilever structures 81, 82 and 83.

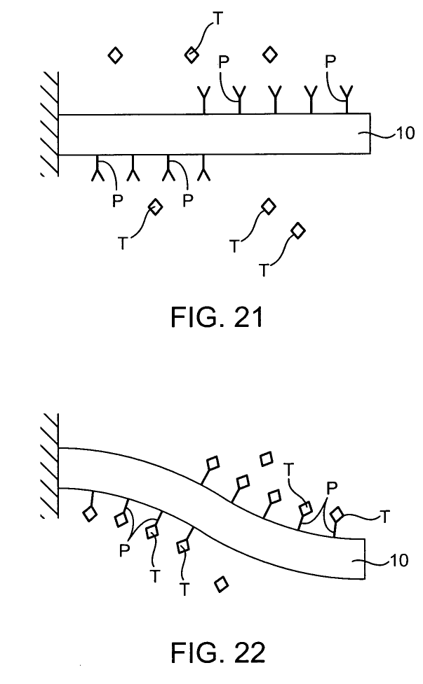

In any of the above described embodiments of the present invention, a surface of the first (or second) material of the bi-material cantilever may be functionalized with a probe substance such that when a target substance binds thereto, a change in surface stress occurs on the surface, thereby causing the cantilever to deflect. For example, referring to FIG. 1, first material 20 may be functionalized with probe molecules. Thus, when cantilever 10A is exposed to target molecules, the cantilever will deflect to the position shown in FIG. 2. FIGS. 21 and 22 show a cantilever 10 made from a single block of material in which a top surface of first portion 12 and a bottom surface of a second portion 14 have been functionalized with a probe substance P. As shown in FIG. 22, when a target substance T binds to probe substance P, the surface stresses on cantilever 10 change, resulting in cantilever deflection. As can be seen, a particular advantage of this design is that laser beam 42 is reflected directly back into laser 40 as cantilever 10 deflects.

What is claimed is:

1. A bi-material cantilever, comprising:
a first portion and a second portion positioned end-to-end, wherein the first portion comprises a first material positioned on top of a second material, and wherein the second portion comprises the second material positioned on top of the first material.

2. The bi-material cantilever of claim 1, wherein the first and second portions of the cantilever are of equal length.

3. The bi-material cantilever of claim 1, wherein the cantilever has first and second ends that remain parallel to one another during deflection of the cantilever.

4. The bi-material cantilever of claim 1, wherein the first and second materials have different thermal expansion coefficients.

5. The bi-material cantilever of claim 1, wherein the first material is nitride.

6. The bi-material cantilever of claim 1, wherein the second material is aluminum.

7. The bi-material cantilever of claim 1, wherein the first material in the first portion of the cantilever abuts the second material in the second portion of the cantilever.

8. The bi-material cantilever of claim 1, wherein the second material in the first portion of the cantilever is continuous with the second material in the second portion of the cantilever.

9. The bi-material cantilever of claim 1, wherein the first material is deposited on the second material by plasma enhanced chemical vapor deposition or by electron beam evaporation.

10. The bi-material cantilever of claim 1, further comprising:
a reflective panel connected to one end of the cantilever.

11. The bi-material cantilever of claim 10, further comprising:
an infra-red radiation absorbing pad connected to the reflective panel.

12. The bi-material cantilever of claim 1, further comprising:
an electrical path disposed along a surface of the bi-material cantilever.

13. The bi-material cantilever of claim 1, wherein a surface of the first material of the bi-material cantilever is functionalized with a probe substance such that when a target substance binds thereto, a change in surface stress occurs on the surface, thereby causing the cantilever to deflect.

14. A structure formed from a pair of bi-material cantilevers, wherein each bi-material cantilever comprises:
a first portion and a second portion positioned end-to-end, wherein the first portion of each bi-material cantilever comprises a first material positioned on top of a second material, and wherein the second portion of each bi-material cantilever comprises the second material positioned on top of the first material, and wherein the pair of bi-material cantilevers are connected together.

15. The structure of claim 14, wherein an end of one bi-material cantilever is connected to an end of the other bi-material cantilever.

16. The structure of claim 14, wherein the first portion of one cantilever is connected to the second portion of the other cantilever.

17. The structure of claim 14, wherein the pair of bi-material cantilevers are connected together at right angles to one another.

18. The structure of claim 14, wherein the pair of bi-material cantilevers are connected together through an intermediate member.

19. The structure of claim 18, wherein the pair of bi-material cantilevers are positioned parallel to one another.

20. The structure of claim 14, further comprising:
a reflective panel connected to an end of at least one of the bi-material cantilevers.

21. The structure of claim 20, wherein the reflective panel is positioned between the bi-material cantilevers.

22. The structure of claim 20, wherein one of the bi-material cantilevers is positioned between the reflective panel and the other bi-material cantilever.

23. The structure of claim 20, further comprising:
an infra-red radiation absorbing pad connected to the reflective panel.

24. The structure of claim 14, wherein a surface of the first material of each of the bi-material cantilevers is functionalized with a probe substance such that when a target substance binds thereto, a change in surface stress occurs on the surface, thereby causing each of the cantilevers to deflect.

25. The structure of claim 14, further comprising:
a third bi-material cantilever comprising a first portion and a second portion positioned end-to-end, wherein the first portion comprises the first material positioned on top of a second material, and wherein the second portion comprises the second material positioned on top of the first material, and wherein the third bi-material cantilever is connected to the pair of bi-material cantilevers.

26. The structure of claim 25, wherein
a reflective panel connected to an end of each of the three bi-material cantilevers.

27. The structure of claim 26, wherein the reflective panel is movable in three degrees of freedom.

28. A -material cantilever, comprising:
a block of material having a top surface and a bottom surface, wherein an equal portion of the top and the bottom surfaces of the cantilever are functionalized with a probe substance, such that when a target substance binds thereto, a change in surface stress occurs on the functionalized portions of the top and bottom surfaces of the cantilever, thereby causing the cantilever to deflect, wherein opposite ends of the cantilever remain parallel to one another during deflection of the cantilever.
